(19) United States
(12) Patent Application Publication
(10) Pub. No.: US 2014/0309609 A1
(43) Pub. Date: Oct. 16, 2014

(54) HEMOSTATIC DRESSING

(71) Applicant: Chen-Ping Hsu, Miaoli County (TW)

(72) Inventor: Chen-Ping Hsu, Miaoli County (TW)

(73) Assignee: CENEFOM CORP., Miaoli County (TW)

(21) Appl. No.: 13/908,985

(22) Filed: Jun. 3, 2013

(30) Foreign Application Priority Data
Apr. 11, 2013 (TW) 102112813

(51) Int. Cl. A61F 13/00; A61L 15/20; A61L 15/24; A61L 15/26

(52) U.S. Cl. CPC A61F 13/00068; A61L 15/26; A61L 15/20; A61L 15/24; A61F 13/00063; A61F 2013/00106; A61F 2013/0028
USPC 604/369

(57) ABSTRACT

A medical dressing for trauma injuries is disclosed. The dressing includes a base, a porous foam layer and a protective film. The base has a muffling side. The porous foam layer is partially attached on the muffling side. The protective film covers the muffling side and the porous foam layer.

### BACKGROUND OF THE INVENTION

1. Technical Field

The invention relates to externally-applied medical supplies, particularly to topical hemostatic tape for wounds.

2. Related Art

A medical dressing is an adjunct used by a person for application to a wound to promote healing and/or prevent further harm. A dressing is designed to be in direct contact with the wound, which makes it different from a bandage, which is primarily used to hold a dressing in place. Dressings are frequently used in first aid and nursing. It is generally used as a tool to stem the flow of traumatic bleeding. Usual dressings such as bandages, gauze and adhesive bandages possess water-absorbability, but they lack a hemostatic effect.

Some medical dressings with a hemostatic effect such as thrombin or fibrin are provided, but their water-absorbability is not good enough to prevent absorbed blood from leaking. Additionally, such a dressing needs a bandage or adhesive tape for fixation. That is inconvenient for users.

There is a tourniquet with a hemostatic layer in the market. Such a tourniquet is a constricting or compressing device, specifically a bandage, used to control venous and arterial circulation to an extremity for a period of time. Pressure is applied circumferentially upon the skin and underlying tissues of a limb; this pressure is transferred to the walls of vessels, causing them to become temporarily occluded. However, the tourniquet is available only for heavy bleeding or bleeding on four limbs. And application of the tourniquet will preclude blood from flowing and make the injured feel uncomfortable.

### SUMMARY OF THE INVENTION

An object of the invention is to provide a hemostatic dressing, which is simple to be applied and is provided with hemostatic effect.

To accomplish the above object, the dressing includes a base, a porous foam layer and a protective film. The base has a muffling side. The porous foam layer is partially attached on the muffling side. The protective film covers the muffling side and the porous foam layer.

### BRIEF DESCRIPTION OF THE INVENTION

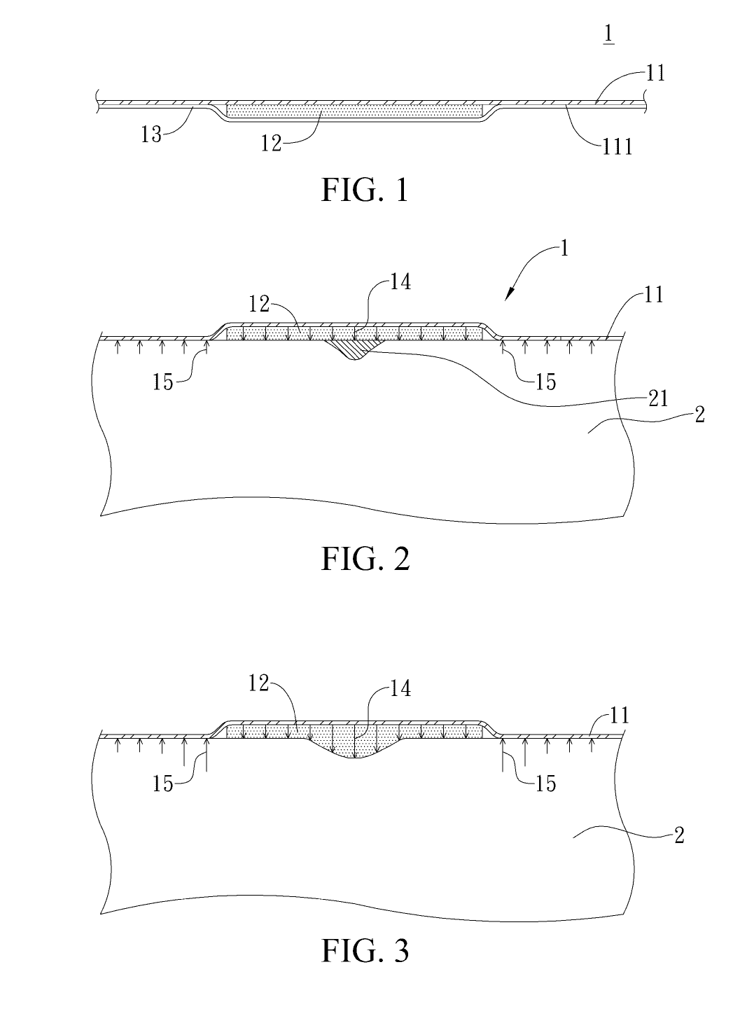

FIG. 1 is a sectional view of the first embodiment of the invention;

FIG. 2 is a schematic view of the invention before water absorption;

FIG. 3 is a schematic view of the invention after water absorption;

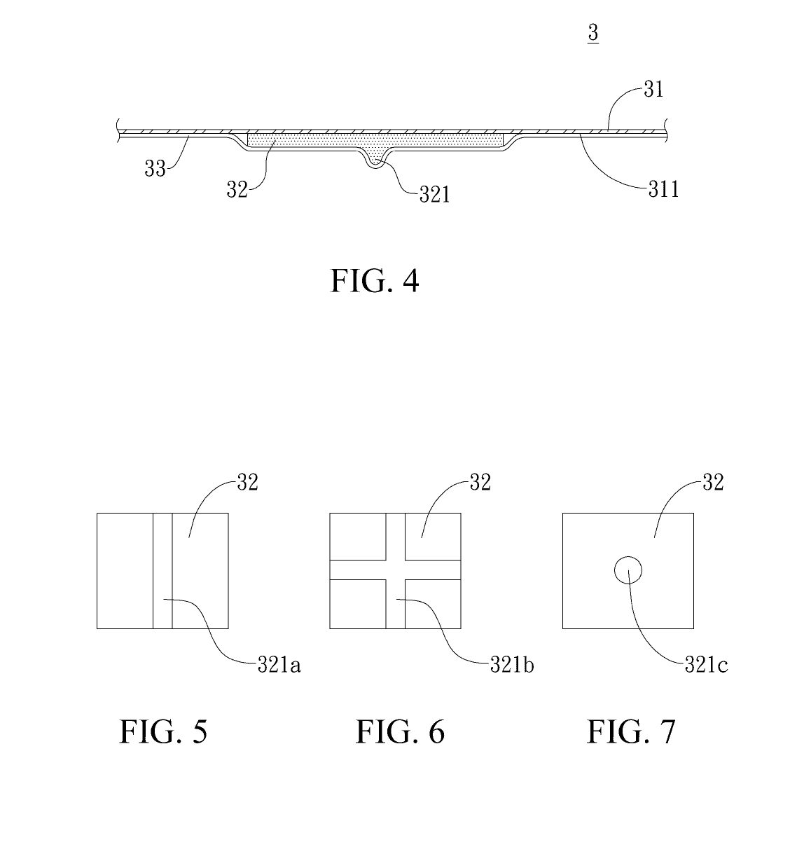

FIG. 4 is a sectional view of the second embodiment of the invention;

FIG. 5 is a schematic view of the third embodiment of the invention;

FIG. 6 is a schematic view of the fourth embodiment of the invention; and

FIG. 7 is a schematic view of the fifth embodiment of the invention.

### DETAILED DESCRIPTION OF THE INVENTION

FIG. 1 shows the first embodiment of the invention. As shown, the dressing 1 includes a base 11, a porous foam layer 12 and a protective film 13. The base 11 has a muffling side 111 for muffling a patient's skin. The porous foam layer 12 is partially attached on the muffling side 111 for absorbing water to generate pressure. The protective film 13 covers the muffling side 111 and the porous foam layer 12 for protecting the muffling side 111 from being stained and preventing the porous foam layer 12 from absorbing moisture before use. The protective film 13 may be release paper.

Preferably, the muffling side of the base 11 is adhesive. The adhesion may come from the property of material or an adhesive coating on the muffling side 11.

The size of the dressing 1 is not limited. The base 11 can be selected from adhesive materials such as breathable tape and adhesive bandage or from non-adhesive materials with an adhesive coating. Alternatively, the base 11 can be disposed with a hook-and-loop fastener strip or other fasteners for connection. The material of the base 11 can be selected from nonwoven polyester fibers, polyurethane, cotton or silk.

The porous foam layer 12 may be formed into different sizes and shapes for matching various wounds and may be made of hydrophilic polymers. To deal with a bleeding, the pressure generated due to absorbing water by the porous foam layer 12 should be ranged from 2 mmHg to 500 mmHg. For purpose of product classification, the pressure can be further configured between 200 mmHg and 500 mmHg and between 2 mmHg and 30 Hg. The porous foam layer 12 may be made of polyvinyl formal (PVF) obtained by formalization of polyvinyl alcohol (PVA). The above indicated polymer materials are compatible with skin such that interfacial adhesion problems can be avoided, and have excellent capability of water absorption such that expansion pressure can be generated and being proportional to the amount of water absorbed.

Please refer to FIG. 2. The dressing 1 must be used on skin 2 before skin treatment. The protective film 13 has to be removed first. The muffling side 111 is attached on the skin 2 and the porous foam layer 12 must completely cloak the wound 21. At this time, the porous foam layer 12 which attaches the wound 21 generates an action force 14 and the muffling side 111 which attaches the skin 2 generates an opposite reaction force 15. As shown in FIG. 3, the porous foam layer 12 will expand after absorbing water and exert a stronger action force 14 against the wound 21 to obtain a hemostatic effect. And the reaction force 15 will become larger. The action force 14 depends upon the amount of water absorbed by the porous foam layer 12. In other words, the more the water is absorbed, the larger the expansion pressure is generated. The total action force 14 is equal to the total reaction force 15. The reaction force 15 will be borne by the skin 2 attached by the muffling side 111. The pressure generated due to absorbing water by the porous foam layer 12 is exerted on the wound 21 only, but not on the circumferential areas of skin 2 around the wound 21. Thus, the present invention avoids the problems of being overly constricted or compressed to wound 21 which may cause insufficient supply of blood to underlying tissue and resulting tissue damage as found in prior arts. Further, the present invention readily applies to various parts of human body, without being restricted to the limb area as occurred in prior arts.

The first embodiment of the present invention provides a topical hemostatic tape 1 to be used in wound 21 of skin 2, wherein its porous foamed layer 12 expands by absorbing water, thus establish suitable hemostatic effect by applying pressure to the wound 21. And, the first embodiment of the present invention applies to various parts of skins of a patient, without being restricted to the limb area as occurred in prior arts, and thus is more convenient, comfortable and efficient, and safer than prior arts.

FIG. 4 shows the second embodiment of the invention. As shown, the dressing 3 includes a base 31, a porous foam layer 32 and a protective film 33. The base 31 has a muffling side 311 for muffling a patient's skin. The porous foam layer 32 is partially attached on the muffling side 311 for absorbing water to generate pressure. The porous foam layer 32 is formed with a projection 321 toward the protective film 33. The projection 321 can attach a wound more fitly. The protective film 33 covers the muffling side 311 and the porous foam layer 32 for protecting the muffling side 311 from being stained and preventing the porous foam layer 32 from absorbing moisture before use.

FIGS. 5-7 depict various shapes of the projection 321. The projection 321a shown in FIG. 5 is of a linear shape. The projection 321b shown in FIG. 6 is of a cross shape. The projection 321c shown in FIG. 5 is of a spot shape. A skilled person in the art must know other shapes of the projection are also available.

It will be appreciated by persons skilled in the art that the above embodiments have been described by way of example only and not in any limitative sense, and that various alterations and modifications are possible without departure from the scope of the invention as defined by the appended claims.

What is claimed is:

1. A hemostatic dressing comprising:
a base having a muffling side;
a porous foam layer, partially attached on the muffling side for absorbing liquid to generate pressure; and
a protective film, covering the muffling side and the porous foam layer.

2. The hemostatic dressing of claim 1, wherein the base is made of nonwoven polyester fibers, polyurethane, cotton or silk.

3. The hemostatic dressing of claim 1, wherein the muffling side is provided with an adhesive.

4. The hemostatic dressing of claim 1, wherein the base is made of breathable tape.

5. The hemostatic dressing of claim 1, wherein the base is provided with a hook-and-loop fastener strip or a fastener.

6. The hemostatic dressing of claim 1, wherein the pressure is between 2 mmHg and 500 mmHg.

7. The hemostatic dressing of claim 1, wherein the porous foam layer is formed with a projection toward the protective film.

8. The hemostatic dressing of claim 7, wherein the projection is of a linear, cross or spot shape.

9. The hemostatic dressing of claim 1, wherein the porous foam layer is made of polyvinyl formal (PVF) obtained by formalization of polyvinyl alcohol (PVA).

10. The hemostatic dressing of claim 1, wherein the porous foam layer is added with a hemostat or medicine.

11. The hemostatic dressing of claim 1, wherein the protective film is release paper.
